(19) United States
(12) Patent Application Publication
(10) Pub. No.: US 2021/0330687 A1
(43) Pub. Date: Oct. 28, 2021

(54) BIFIDOGENIC HYPOALLERGENIC GOS COMPOSITIONS AND METHODS FOR PROVIDING THE SAME INVOLVING BETA-GALACTOSIDASE FROM A STRAIN OF LACTOBACILLUS DELBRUECKII SSP BULGARICUS

(71) Applicant: FrieslandCampina Nederland B.V., Amersfoort (NL)

(72) Inventor: Linqiu Cao, Wageningen (NL)

(21) Appl. No.: 17/273,448

(22) PCT Filed: Sep. 4, 2019
(86) PCT No.: PCT/EP2019/073519

§ 371 Date: Mar. 4, 2021

(30) Foreign Application Priority Data
Sep. 6, 2018 (EP) 18193000.9

(51) Int. Cl. A61K 31/702; C12P 19/12; C12P 19/14; A23L 33/125; A23L 33/00

(52) U.S. Cl. CPC A61K 31/702; C12P 19/12; A23V 2002/00; A23L 33/125; A23L 33/40; C12P 19/14

(57) ABSTRACT

The invention relates to the field of hypoallergenic oligosaccharides for use in nutritional compositions, in particular to oligosaccharides having prebiotic properties. Provided is a hypoallergenic oligosaccharide composition comprising galacto-oligosaccharides (GOS), wherein (i) the galacto-oligosaccharides (GOS) content is at least 40% by weight of the total dry matter of the composition; (ii) the allolactose content is at least 10% by weight of the total dry matter of the composition; (iii) the 6′-GL content is at least 30% by weight of the total GOS in the composition; and (iv) at least 0.5% by weight of the total GOS has a polymerization degree (DP) of six or more. The GOS composition does not trigger GOS-allergy as assessed in a basophil activation test (BAT).

The invention relates to the field of oligosaccharides for use in nutritional compositions, in particular to oligosaccharides having prebiotic properties. Products having prebiotic properties can promote a healthy flora in the gastrointestinal tract of humans and/or animals. Typically, the products induce an enhanced immune function and an improved absorption of minerals like calcium, iron and magnesium, which is beneficial to menopausal woman, elderly persons, and patients suffering from a disturbed intestinal function.

The human gastrointestinal tract (GIT) hosts a large bacterial population of 500-1000 different phylotypes that reside in the colon. Among them, Bifidobacterial species are the predominant microbial in the infant GIT, exerting beneficial effects to their host such us immuno-stimulation, human pathogen inhibition, vitamin production, and anticarcinogenic activity, among others (Harmsen, H. J., et al. 2000 J Pediatr Gastroenterol Nutr 30:61-7; Casci, T., et al. 2007 Human Gut microflora in Health and Disease: Focus on Prebiotics. In Functional food and Biotechnology. Ed Taylor and Francis, pp 401-434). Products having a “bifidogenic” effect specifically enhance the growth of bifidobacteria in the intestines. In infants, the enrichment of bifidobacteria makes it more similar to the flora of breast-fed infants and/or can be used to prevent and/or treat any disturbance in the naturally occurring flora in the gastrointestinal tract. These effects are especially beneficial in clinical patients and in newborns.

It is a well-known fact that human milk, in addition to providing nutrients and energy necessary for babies to thrive, also contains non-digestible oligosaccharides (human milk oligosaccharides; HMOs). The HMOs promote the colonization of microbiota, like bifidobacteria and lactobacilli, in the small intestine, thus establishing gut microflora with many health benefits, including increased resistance to diarrhoea and infections, maturing the immune system and stimulating immune system activity.

It is also known that the gut microflora of formula-fed infants differs from that of the breastfed infants. In general, the microbiota of breast-fed infants mainly contains bifidobacteria, while the microbiota of formula-fed infants is more diverse, with bifidobacteria often being the predominant species, but also containing other and less beneficial species in substantial amounts. This is presumably due to the lack of certain non-digestible HMOs in infant formulae, which act as prebiotics and thus contribute to the bifidogenic microbiota.

For better bifidogenic efficacy, most current infant formulas contain galactooligosaccharides (GOS). GOS are carbohydrate components that are not digestible by humans, but which have been shown to have a growth-promoting effect on bifidobacteria and lactobacilli, as they are able to ferment them. Moreover, GOS have been investigated as potential anti-inflammatory agents against IBD and IBS. In some formulas, GOS are combined with live intestinal bacteria for better bifidogenicity (synbiotics), see for example WO00/33854. In the past decade, GOS have had an increasing application in human food products, including dairy products, sugar replacements and other nutritional or nutraceutical supplements.

Typically, the basic structure of GOS includes a glucose residue at the reducing end which is elongated typically with up to seven galactose residues (degree of polymerization (DP) of up to 8).

It has been suggested in the art that 6′-galactosyl-lactose (6′-GL) is one of the more important HMOs. See Newburg et al. ((2016), J. Nutri. 146, 358-367), who observed that the three galactosyllactoses (3′-GL, 4′-GL, and 6′-GL) expressed in colostrum galactosyllactose attenuated NF-κB inflammatory signaling in human intestinal epithelial cells and in human immature intestine. This implies that galactosyllactoses may serve as strong physiologic anti-inflammatory agents in human colostrum and early milk, contributing to innate immune modulation.

GOS can be produced by known chemical methods, but the preferred method to synthesize them is the enzymatic approach. Commercial GOS preparations are generally produced via a transgalactosylation reaction by enzymatic treatment of lactose with ß-galactosidases (EC.3.2.1.23) from different sources such as fungi, yeast and/or bacteria, yielding a mixture of oligomers with varied chain lengths, resulting in the formation of a mixture containing approximately 100 different types structures with varying DP and linkages. Beta-Galactosidase is produced in many microorganisms such as Bacillus circulans, Aspergillus oryzae, Kluyveromyces marxianus, Kluyveromyces fragilis, Sporobolomyces singularis, and Lactobacillus fermenturn. GOS structural diversity depends on the enzyme used in the trans-galactosylation reaction, and the reaction conditions such as pH, temperature and enzyme dosage (Dumortier, V., et al. 1990, Carbohydr Res 201: 115-23).

Beta-galactosidases differ in their three-dimensional structures, resulting in stereo- and regioselectivity of glycosidic bonds. For example, typically fungal species such as Aspergillus predominantly produce ß1-6 bonds (thus resulting in mainly 6′-GOS , with 3′-GOS and 4′-GOS as the minor GOS components), while bacteria such as Bacillus predominantly produce ß1-4 bonds (resulting in mainly 4′-GOS). Moreover, beta-galactosidase produced by B. circulans possesses particularly strong transgalactosylation activity, and thus, GOS prepared by beta-galactosidase from B. circulans is commercialized worldwide. Since its introduction to the market (1999), approximately 100 millions of infants have consumed infant formula containing GOS prepared by B. circulans. It has been proven to be a safe ingredient, with a GRAS status acknowledged by the FDA. Moreover, a cohort study on the baby's feces microbiota composition has shown that the feces of infant fed with IF containing GOS resembles that of breast-fed babies (Knol et al. J Ped Gastr Nut 2005, 40:36-42).

In the past few years, however, a small number of very rare cases of GOS-related allergy has been reported in South East Asia. Research has shown that certain oligosaccharide structures present in GOS can exert an allergic response in very sensitive subjects (Chiang, W. C. et al. (2012) J. Allergy Clin. Immunol. 130, 1361-1367). Kaneko et al. (Biosc. Biotechnol. Biochem. 2014, 78, 100-108) observed that GOS produced by treating lactose with a beta-galactosidase preparation derived from B. circulans may induce allergic reactions and revealed that the allergies were caused by two tetrasaccharides [Gal ß1-4 (Gal ß1-4 Gal ß1-6) Glc, Gal ß1-4 Gal ß1-4 Gal ß1-3 Glc]. These GOS allergy cases occurred in subjects who already had a history of atopy, implying that the primary triggers for GOS allergy are something else.

The present inventors aimed at the manufacture of a novel oligosaccharide composition having a high GOS content, and comprising GOS species that possess a desirable combination of prebiotic and hypoallergenic properties. In particular, they sought to provide a GOS preparation having enhanced bifidogenic properties combined with a reduced capacity to induce an allergic response in a subject, e.g. as compared to GOS obtained by Bacillus circulans beta-galactosidase.

To that end, they set out to screen a number of lactic acid-producing bacterial strains for their application in the enzymatic GOS manufacture. This resulted in the identification of beta-galactosidase from specific Lactobacillus delbrueckii strains and the provision of a novel oligosaccharide composition comprising GOS, characterized among others in (i) a high GOS content; (ii) a high 6′-GL content; and (iii) a high content of GOS DP6 or more. Surprisingly, this composition (also herein referred to as “L-GOS”) exhibits strong bifidogenic effects and no detectable response in a BAT-assay, which is indicative of no or very low allergenicity.

Accordingly, in one embodiment the invention provides an oligosaccharide composition comprising galacto-oligosaccharides (GOS), wherein:
(i) the galacto-oligosaccharides (GOS) content is at least 40% by weight of the total dry matter of the composition;
(ii) the allolactose content is at least 10% by weight of the total dry matter of the composition;
(iii) the 6′-galactosyl-lactose (6′-GL) content is at least 30% by weight of the total GOS in the composition; and
(iv) at least 0.5% by weight of the total GOS has a polymerization degree (DP) of six or more.

As used herein, the term “GOS” refers to non-digestible oligosaccharides comprised of 1 to 7 molecules of galactose and 1 molecule of glucose as the reducing end. In some cases, galactobiose or branched GOS can be formed. However, whenever in the present application reference is made to the content of a given oligosaccharide relative to the total GOS content, or the GOS content based on dry matter, allolactose is not included in the total GOS content. This is for the reason that allolactose could, historically, not be distinguished from lactose in quantitative HPLC measurement that is defined in AOAC GOS determination (AOAC method February 2001). Hence, the expression “by weight of the total GOS” or “GOS content based on dry matter” refers to GOS-compounds including 6′-GL but excluding allolactose.

Among others, an oligosaccharide composition of the invention is characterized by a relatively high GOS content when compared to known GOS compositions obtained by transgalactosylation. In one embodiment, the GOS content is at least 42% by weight, preferably at least 44%, more preferably at least 46% and most preferably at least 48% by weight of the total dry matter of the composition. In a further embodiment, the GOS content is at least 50%, preferably at least 55%, more preferably at least 60% by weight of the total dry matter of the composition

At least 0.5% by weight of the total GOS in an oligosaccharide composition as provided herein has a DP of six or more. This includes one or more of DP6, DP7, DP8 and DP9, preferably at least DP6 and/or DP7. As disclosed in WO2008/041843, GOS pentasaccharides (herein also referred to as DP5) and GOS hexasaccharides (DP6), are effective anti-Ctx-B adhesives by preventing Ctx binding to its natural receptor GM1 on a target cell. Herewith, the presence of DP6 can contribute to the treatment or prevention of an acute or chronic disease associated with or caused by the adhesion and/or uptake of a cholera toxin family member, in particular diarrhoeal diseases. Besides, the presence of DP>5 GOS components will be mainly utilized by Bifidobacteria longum, which is one of the major bifidobacterial species in infant gut microbiota, thus stimulating not only the growth of a balanced gut bifidobacterial species (Barboza M et al., (2009) Applied and Environmental Microbiology 75:7319-7325) but also conferring the infant with reducing incidence of influenza and fever (Namba et al. (2010) Biosci Biotechnol Biochem. 74:939-45) and fewer respiratory infections (Puccio et al. (2007) Nutrition 23:1-8).

In one aspect, the DP≥6 content is at least 1% by weight, preferably at least 1.5% by weight. For example, the content of DP6+DP7 GOS is in the range of 0.8-3 wt %, like 1.0-2.5 wt % or 1.1-2.8 wt %. Compositions with higher contents of DP6+DP7 GOS are also envisaged. For example, after the purification, the GOS weight percentage may increase up to 1.5-2.0 fold due to the removal of lactose and mono-sugars, like glucose and galactose. Accordingly, in one embodiment the content of DP6+DP7 GOS is in the range of 1.2-6 wt %, like 1.2-5 wt % or 1.4-4 wt %.

Allolactose is a disaccharide similar to lactose. It consists of the monosaccharides D-galactose and D-glucose linked through a ß1-6 glycosidic linkage instead of the ß1-4 linkage of lactose. It may arise from the occasional transglycosylation of lactose by ß-galactosidase. Allolactose is an inducer of the lac operon, which allows the lactose transport and digestion in E. coli and many other enteric bacteria. Its presence is crucial for the induction of beta-galactosidase responsible for lactose and GOS utilization when there is no glucose available. Therefore, we surmise that the allolactose is an important component of GOS. The allolactose content of a composition of the invention is at least 10% by weight of the total dry matter of the composition. In one embodiment, the allolactose content is at least 12%, preferably at least 13% by weight of the total dry matter of the composition. Typically, the allolactose content is not more than 20 wt %, like up to 18, 16 or 15 wt % on GOS.

The GOS trisaccharide 6′-galactosyllactose is known to have an effect of stimulating growth of Bifidobacterium or Lactobacillus present in human large intestines, and thus is employed in foods for infants and elderly people, such as foods for improving bowel movement or diarrhea prevention, and the like. In addition, galactosyllactoses are known to have an effect of inhibiting the rate of skin aging by promoting behavior of large intestine, which is assumed to be induced by smooth bowel activity through changing microflora in large intestines (an effect of stimulating growth of enteric beneficial bacteria), thereby inhibiting skin aging. The 6′-galactosyl-lactose (6′-GL) content of a composition provided herein is at least 30% by weight of the total GOS in the composition. For example, it is at least 32 wt %, 34 wt %; 36 wt %, or at least 38 wt %. Preferably, the 6′-GL content is at least 40 wt %, more preferably at least 42 wt %, 43 wt % or 44 wt % of the total GOS in the composition.

As will be understood, any of the preferred embodiments of features (i) through (iv) recited herein above can be combined which each other in any combination.

In a specific embodiment, the invention provides an oligosaccharide composition according to any one of the preceding claims wherein

(i) the GOS content is at least 65%, preferably at least 70% by weight of the total dry matter of the composition;

(ii) the allolactose content is at least 12% by weight of the total dry matter of the composition;

(iii) the 6′-GL content is at least 40% by weight of the total GOS in the composition; and

(iv) at least 1% by weight of the total GOS is DP≥6.

The invention also relates to a method for providing an oligosaccharide composition according to the invention comprising the steps of (i) contacting a lactose feed with a beta-galactosidase (EC 3.2.1.23) and (ii) allowing for oligosaccharide synthesis, wherein said beta-galactosidase is derived from Lactobacillus delbrueckii subspecies bulgaricus or Lactobacillus delbrueckii subspecies lactis. For instance, the method comprises subjecting whey permeate or lactose to enzymatic transgalactosylation using beta-galactosidase. Conditions for the transgalactosylation reaction are known in the art. For example, GOS synthesis is suitably performed by adding the selected beta-galactosidase to a lactose suspension of at least 40% (w/w) lactose in dry matter that has been pre-adjusted with desired pH at 50-60° C. The enzyme dosage used is strongly dependent on the lactose concentration, pH and temperature. However, the enzyme dosage chosen should be sufficient to clarify the lactose suspension within the time selected. Typically, the following conditions can be applied:

50% lactose, pH6.5, temperature 50° C., and enzyme dosage 3 LU/gram lactose, the reaction time at least 48 hours.

In particular, it was found that a beta-galactosidase having an amino acid sequence according to SEQ ID NO:1 (see FIG. 7A), or a sequence that is at least 90% identical thereto, is capable of providing an oligosaccharide of the invention characterized by a high GOS content, strong bifidogenic properties and low allergenicity. This enzyme is structurally distinct from those found in the strains used by Vasiljevic et al. (Lait 83 (2003), 453-467) which may explain the fact that the formation of penta- or hexasaccharides was not detected in any of their processes.

The enzyme used by Vasiljevic et al. (DSM20081; synonym: ATCC 11842) was also used by Nguyen et al. (J. Agric. Food Chem. 2012, 60, 1713-1721). Also in the latter document, the formation of penta- or hexasaccharides was not reported.

In one embodiment, the beta-galactosidase has an amino acid sequence that is at least 92%, 93%, 94%, 95%, 96%, 97% or 98% identical to SEQ ID NO:1. For example, the enzyme shows at least 99%, 99.3%, 99.5%, 99.6% or 99.8% sequence identity to SEQ ID NO:1.

The difference in the amino acid sequence is acceptable as long as the beta-galactosidase activity is maintained (the activity may be varied to a degree). As long as the conditions are satisfied, the position of the difference in the amino acid sequence is not particularly limited, and the difference may arise in a plurality of positions. The difference of the amino acid sequence may arise in a plurality of positions. Preferably, the equivalent protein is obtained by causing conservative amino acid substitution in an amino acid residue which is not essential for beta-galactosidase activity. The term “conservative amino acid substitution” means the substitution of an amino acid residue with another amino acid residue having a side chain with similar properties.

Amino acid residues are classified into several families according to their side chains, such as basic side chains (for example, lysine, arginine, and histidine), acidic side chains (for example, aspartic acid and glutamic acid), uncharged polar side chains (for example, glycine, asparagine, glutamine, serine, threonine, tyrosine, and cysteine), nonpolar side chains (for example, alanine, valine, leucine, isoleucine, proline, phenylalanine, methionine, and tryptophan), ß-branched side chains (for example, threonine, valine, and isoleucine), and aromatic side chains (for example, tyrosine, phenylalanine, tryptophan, and histidine). Conservative amino acid substitution is preferably the substitution between amino acid residues in one family. In one embodiment, the equivalent enzyme has the amino acid sequence of SEQ ID NO:1 with up to 6, preferably up to 5, more preferably up to 4 non-conservative amino acid substitutions.

An enzyme for use in the present invention having the above-described amino acid sequence is readily prepared by a genetic engineering technique. For example, an appropriate host cell (for example, Escherichia coli) is transformed by a DNA encoding the present enzyme. The DNA may have a nucleic acid sequence identical or equivalent to the nucleic acid molecule according to SEQ ID NO: 2 (see FIG. 7B). The “equivalent nucleic acid sequence” herein denotes a nucleic acid sequence which is partly different from the nucleic acid sequence according to SEQ ID NO: 2, but in which the function (herein, ß-galactosidase activity) of the protein encoded by the sequence is not substantially affected by the difference.

After protein expression, the protein expressed in the transformant is collected, and thereby preparing the present enzyme. The collected protein is treated as appropriate according to the intended use. The enzyme thus obtained as a recombinant protein may be subjected to various modifications. For example, the enzyme composed of a recombinant protein linked to any peptide or protein can be obtained by producing a recombinant protein using a vector into which a DNA encoding the enzyme has been inserted together with other appropriate DNA. In addition, modification for causing addition of a sugar chain and/or a lipid, or N- or C-terminal processing may be carried out. These modifications allow, for example, extraction of a recombinant protein, simplification of purification, or addition of biological functions.

In a preferred embodiment, however, the enzyme for use in a method of the present invention is comprised in a micro-organism which endogenously expresses the enzyme. This allows cheaper and easier processing as it saves the effort of isolating the enzyme. The micro-organism, e.g. strain of Lactobacillus delbrueckii subspecies bulgaricus, may be used as whole cells or as active part or fraction thereof, preferably a cell free extract.

A strain of Lactobacillus delbrueckii subspecies bulgaricus capable of producing a galactosidase enzyme activity for use in providing an oligosaccharide composition of the invention has been deposited under accession number DSM20080.

For reasons outlined herein above, an oligosaccharide composition according to the invention can have various beneficial effects on the human or animal body upon oral ingestion. In particular, the compositions of the invention may provide their health-promoting action throughout the entire small and large intestine and/or one or more parts thereof, including the duodenum, jejunum, ileum and colon. Similarly, the compositions of the invention may also provide their anti-adhesion and/or their bifidogenic effect throughout the entire intestinal tract and/or parts thereof, which may be the same or different parts.

Accordingly, the invention also provides a nutritional composition comprising an oligosaccharide composition as herein disclosed. As used herein, a “nutritional composition” includes one or more of protein, carbohydrate, lipid source, one or more vitamins, one or more minerals, etc.

Hence, it also encompasses food supplements which may not have protein, lipid and/or carbohydrate sources. As used herein, a nutritional composition refers to any composition or formulation that goes into the alimentary canal for nutritional purposes, in whatever solid, liquid, gaseous state. Thus, a nutritional composition can be a food item or a drink item.

In one embodiment, the nutritional composition comprises a protein source, a lipid source, a carbohydrate source, and an oligosaccharide composition according to the invention. For example, the nutritional composition comprises fat, protein, carbohydrate, vitamins and minerals, all of which are selected in kind and amount to provide a sole source of nutrition for the targeted or defined (human) population. The nutritional composition is preferably selected from the group consisting of an infant formula, follow-up formula, growing-up milk, a dairy product, a cereal product and a medical nutritional product. Medical nutrition products are available as enteral formulas ingested both orally, for example as beverages, foods or supplement-like formats, and via intubation.

In one aspect, the nutritional composition is an infant formula formulated for an infant of between 0 and 6 months of age, between 3 and 6 months of age, 6 and 9 months of age or 9 and 12 months of age. Infant formulas for use as base formulas include any known ready-to-feed infant formula, or any nutritional formula suitable for use in infants, provided that such a formula is a sole source nutritional having caloric density and osmolality values within the ranges defined herein. Many different sources and types of carbohydrates, fats, proteins, minerals and vitamins are known and can be used in the base formulas herein, provided that such nutrients are compatible with the added ingredients in the selected formulation and are otherwise suitable for use in an infant formula. Carbohydrates suitable for use in the base formulas herein may be simple or complex, lactose-containing or lactose-free, or combinations thereof, non-limiting examples of which include hydrolyzed, intact, naturally and/or chemically modified cornstarch, maltodextrin, glucose polymers, sucrose, corn syrup, corn syrup solids, rice or potato derived carbohydrate, glucose, fructose, lactose, high fructose corn syrup and further indigestible oligosaccharides such as fructooligosaccharides (FOS), and combinations thereof. Particularly preferred is an infant formula comprising the combination of sialyllactose and an oligosaccharide composition of the invention comprising GOS.

Proteins suitable for use in the base formulas herein include hydrolyzed, partially hydrolyzed, and non-hydrolyzed or intact proteins or protein sources, and can be derived from any known or otherwise suitable source such as milk (e.g., casein, whey, human milk protein), animal (e.g., meat, fish), cereal (e.g., rice, corn), vegetable (e.g., soy), or combinations thereof. In one embodiment, the composition of the invention comprises a whey fraction comprising the whey proteins a-lactalbumin (a-LA) and casein macropeptide (CMP), wherein the weight ratio between a-LA and CMP is <2.

Proteins for use herein can also include, or be entirely or partially replaced by, free amino acids known for or otherwise suitable for use in infant formulas, non-limiting examples of which include alanine, arginine, asparagine, carnitine, aspartic acid, cystine, glutamic acid, glutamine, glycine, histidine, isoleucine, leucine, lysine, methionine, phenylalanine, proline, serine, taurine, threonine, tryptophan, taurine, tyrosine, valine, and combinations thereof. These amino acids are most typically used in their L-forms, although the corresponding D-isomers may also be used when nutritionally equivalent. Racemic or isomeric mixtures may also be used.

The lipid source in a composition according to the invention may be any type of lipid or combination of lipids which are suitable for use in (children's) nutritional products. Examples of suitable lipid sources are tri, di, and monoglycerides, phospholipids, sphingolipids, fatty acids, and esters or salts thereof. The lipids may have an animal, vegetable, microbial or synthetic origin. Of particular interest are polyunsaturated fatty acids (PUFAs) such as gamma linolenic acid (GLA), dihomo gamma linolenic acid (DHGLA), arachidonic acid (AA), stearidonic acid (SA), eicosapentaenoic acid (EPA), docosahexaenoic acid (DHA), docosapentaenoic acid (DPA) and conjugated linoleic acid (CLA). CLA is important in the protection against eczema and respiratory diseases in children. This particularly involves the cis-9, trans-11 and cis-12 isomers of CLA. Examples of suitable vegetable lipid sources include sun flower oil, high oleic sun flower oil, coconut oil, palm oil, palm kernel oil, soy bean oil, etc. Examples of suitable lipid sources of animal origin include milkfat, for example anhydrous milkfat (AMF), cream, etc. In a preferred embodiment, a combination of milkfat and lipids of vegetable origin is used.

Vitamins and similar other ingredients suitable for use in a nutritional composition include vitamin A, vitamin D, vitamin E, vitamin K, thiamine, riboflavin, pyridoxine, vitamin B12, niacin, folic acid, pantothenic acid, biotin, vitamin C, choline, inositol, salts and derivatives thereof, and combinations thereof. Suitable minerals include calcium, phosphorus, magnesium, iron, zinc, manganese, copper, chromium, iodine, sodium, potassium, chloride, and combinations thereof.

In view of the surprisingly low allergenicity of an oligosaccharide composition of the invention, the invention also provides a nutritional composition comprising (i) an oligosaccharide composition of the invention and (ii) at least one further ingredient selected from the group consisting of a hypoallergenic or non-allergenic protein source, preferably a non-allergenic milk protein hydrolysate, free amino acids, probiotics, a lipid source, and carbohydrates, such as lactose, saccharose, starch or maltodextrin.

Hypoallergenic or non-allergenic protein sources are known in the art, particularly for employment in infant formula. In one embodiment, the at least one further hypoallergenic or non-allergenic ingredient is selected from non-allergenic protein hydrolysates and hydrolysates substantially free of allergenic proteins, hypoallergenic protein sources, and hydrolyzed whey proteins. The terms non-allergenic hydrolysates and hydrolysates substantially free of allergenic proteins as used herein are interchangeable. They refer to protein hydrolysates that can be administered to infants having intolerance against dietary proteins, more particularly cow's milk proteins, without inducing allergic reactions. For example, U.S. Pat. No. 5,039,532 discloses a hydrolyzed whey protein material from which allergens consisting of alpha-lactalbumin, beta-lactoglobulin, serum albumin and immunoglobulins have not been removed and wherein the hydrolyzed protein material including hydrolyzed allergens is in a form of hydrolysis residues having a molecular weight not above 10,000 Da so that the hydrolyzed material is substantially free from allergenic proteins and allergens of protein origin. In one embodiment, a low-allergenic casein hydrolysate with peptides of maximally 3000 Da is included.

In a particular embodiment, the composition is for administration to subjects, in particular infants, at risk of developing allergy, especially cow's milk protein allergy (CMA). Infants that are known to be at risk of developing allergy include infants born from at least one parent who suffers from, or has suffered from, an atopic disorder (e.g. eczema) and/or an allergy, most in particular from CMA.

Given the unexpected bifidogenic properties of an oligosaccharide composition provided herein, the invention also relates to the use of an oligosaccharide composition or a nutritional composition according to the invention, in a method of promoting gut microbiota balance and health, the method comprising administering an effective amount of the oligosaccharide composition or the nutritional composition to an individual in need of such treatment. Hence, also provided is method for promoting gut microbiota balance and health, the method comprising administering to an individual in need of such treatment an effective amount of the oligosaccharide composition or a nutritional composition according to the invention. For example, promoting gut microbiota and health may comprises enhancing bifidogenic micro-organisms in the intestinal tract. In one embodiment, promoting gut microbiota balance and health encompasses improving patient tolerance to various medical treatments that lead to gastrointestinal tract disorders, such treatments including radiotherapy, chemotherapy, gastrointestinal surgery, anesthesia, the administration of antibiotics, analgesic drugs, or treatments for diarrhea.

The invention also provides the use of an oligosaccharide composition or a nutritional composition according to the invention as prebiotic composition, preferably as bifidogenic composition. A still further embodiment of the invention relates to a process for producing a bifidogenic infant or dietetic food, comprising adding an oligosaccharide composition according to the invention to one or more components selected from the group consisting of fats, carbohydrates, minerals, trace elements and vitamins.

The composition may, in addition to the bifidogenic oligosaccharide composition of the invention, contain further prebiotics, as well as prebiotic compounds, in particular fibres and proteins. Fibres in particular include soluble and insoluble non-digestible polysaccharides, such as non-starch polysaccharides (of the cellulose, hemicellulose and other types), resistant starch, gums etc. It is particularly preferred that the compositions of the invention comprise other non-digestible oligosaccharides, which are usually soluble, such as fructo-oligosaccharides (FOS), xylo-oligosaccharides (XOS) and manno-oligosaccharides. These other oligosaccharides are preferably obtained from natural sources, either by direct extraction, e.g. in the case of inulin (FOS), or by hydrolysis of suitable polysaccharide or polysaccharide mixture, e.g. in the case of inulin and levan (FOS), and xylans and other hemicellulose constituents (XOS). The amounts of other oligosaccharides may vary, e.g. from 10% to 400% with respect to the total amount of non-digestible oligosaccharides.

In one embodiment, the composition further comprises one or more human milk oligosaccharides (HMOs). HMOs are well known to the person skilled in the art. In a preferred embodiment the composition comprises one or more HMOs selected from the group consisting of 2′-FL (2′-fucosyl lactose), 3-FL (3-fucosyl lactose), 3′-SL (3′-sialyllactose, 6′-SL (6′-sialyl lactose), LNT (lacto-N-tetraose) and LnNt (lacto-N-neotetraose).

The compositions may advantageously also contain probiotic organisms e.g. at levels of at least 10⁷ viable micro-organisms per daily dose per individual. Probiotic bacteria are known in the art. Suitably, the probiotic is included in the present composition in an amount of 10exp2-10exp13 cfu per g dry weight of the composition, suitably 10exp5-10exp12 cfu/g, most suitably 10exp7-10exp10 cfu/g. Preferably, the probiotic bacteria are not genetically modified. Suitable probiotic bacteria include bacteria of the genus Bifidobacteria (e.g. B. breve, B. longum, B. infantis, B. bifidum), Lactobacillus (e.g. L. Acidophilus, L. paracasei, L. johnsonii, L. plantarum, L. reuteri, L. rhamnosus, L. casei, L. lactis), and Streptococcus (e.g. S. thermophilus). B. breve and B. longum are especially suitable probiotics. Suitable B. breve strains may for example be isolated from the faeces of healthy human milk-fed infants. Other preferred probiotics for use in an infant formula include those capable of promoting the development of an early bifidogenic intestinal microbiota, e.g. the strains disclosed in EP 1974734.

### LEGEND TO THE FIGURES

FIG. 1: HPLC chromatogram of (panel A) a reference GOS+6′-GL and (panel B) a representative L-GOS composition of the invention. For peak identification see Table 1.

FIG. 2: Comparison of GOS Dionex pattern synthesized by whole cells of the strains RFC-219, RFC-227, RFC-302.

FIG. 3: Comparison of GOS profile by using cell-free extract and whole cells of Lactobacillus strain RFC227.

FIG. 4: Comparison of 5 feces bifidobacterial growth using the L-GOS of the invention, sugar control or reference GOS1 and 2 as the only carbon sources in MRS medium. Panel A: after 7 hours of fermentation. Panel B: after 24 hours of fermentation.

FIG. 5: Basophil activation in 4 test subjects (panel A-D) as measured by expression of the basophil activation marker CD203c (MF1=Mean Fluorescence). Different concentrations of test composition (L-GOS) and reference composition (vGOS) were included in the study. For details see Example 5.

FIG. 6: Basophil activation in 4 test subjects (panels A-D) as measured by expression of the basophil activation marker CD36 (MF1=Mean Fluorescence). Different concentrations of test composition (L-GOS) and reference composition (vGOS) were included in the study. For details see Example 5.

FIG. 7: (panel A) Amino acid sequence (SEQ ID NO:1) and (panel B) nucleotide sequence (SEQ ID NO:2) of an exemplary beta-galactosidase enzyme for use in the present invention.

### EXPERIMENTAL SECTION

### Example 1: Preparation of Beta-Galactosidase Enzyme from Lactobacillus

Three selected in house Lactobacillus test strains RFC 219, RFC 227 and RFC 302 were inoculated in MRS media and were grown to an optical density at 600 nm (O.D.₆₀₀) of ˜1.0, and subsequently the inoculates were diluted in fresh MRS medium to an O.D.₆₀₀ of 0.01-0.02 and to grow to OD of ˜1.0-1.5 at 37° C. after 16-32 hours fermentation under aerobic conditions.

Whole cells were harvested by centrifugation of the fermentation broth at 6000 rpm and 18° C. for 10 minutes. After decanting the fermentation broth, two washing steps were performed by repeatedly dispersing the whole cells in demineralized water and centrifugation, aiming to remove any insoluble residues.

The obtained wet whole cells were dispersed in 10 mM natrium citrate buffer, pH6.5 by a ratio of 10% (w/w). The whole cell dispersions were used directly for

GOS synthesis (whole cells) or were disrupted (cell free extract) by a min-bead beater (Biospec Product) using 0.1 mm glass beads at a maximal speed.

Due to the heat generation during the homogenization process, the homogenization process needs to be stopped after 60 seconds. Subsequently, the samples of whole cells were cooled down to 0° C. by immersing in the ice water bath before repeating the homogenization process for second round.

The cell debris after second round homogenization was removed by centrifugation and the cell-free extract (supernatant) was used for GOS synthesis directly without any further treatment.

### Example 2: Enzymatic GOS Synthesis

GOS synthesis was performed under the following conditions:

27 gram lactose crystals (28.42 gram lactochem®, pharma grade, containing 95% lactose) was added to 27 gram 10 mM citrate buffer, pH6.5, which contains beta-galactosidase as a whole cells dispersion (in 10 mM sodium citrate buffer) of Example 1 or the cell-free extract originating from same amount of whole cells dispersion of Example 1, in order to facilitate the comparison. The reaction mixture was stirred using a magnetic stir and the temperature was regulated by water bath to be 50° C. The reaction time was 65 hours.

The amount of enzyme activity needed is pre-determined in an assay by the clarification time of the reaction mixture under the same conditions as above but in ¼ of the above scale, starting from a lactose slurry. Subsequently, the activity of the enzyme preparations, was estimated using the following equation, which was prepared on the basis of a reference enzyme Biolacta N5 (Amano):

Enzyme dosage (Unit/gram lactose)=36.77*(clarification time (hour)\hat{ }−0.549.

For whole cells, the enzyme dosage was calculated to be 2.95 LU/gram lactose for RFC227, 3.3 LU/gram for RFC219 and 4.4 LU/gram lactose for RFC302. As is known by a person skilled in the art, the reaction time can be shortened by adding more enzyme at any moment of the reaction, in order to boost the reaction.

### Example 3: Characterization of GOS Compositions

The content of different oligosaccharide was analyzed by Dionex HPAEC-PAD chromatography (van Leeuwen et al., Carbohydrate Research 2014, 400:59-73) on a analytic CarboPac PA-1 column. The GOS content was estimated by the peak percentage. The validity of this method was confirmed by the reference composition of the commercial Vivinal ®GOS (Table 2).

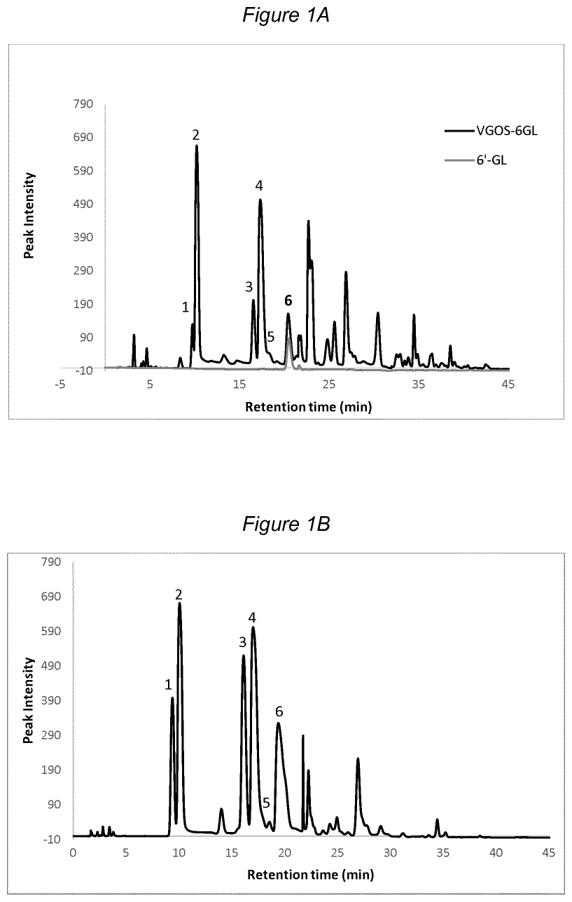

6′-GL component was identified by spiking a reference GOS with 6′-GL standard. As shown in FIG. 1A, 6′-GL is peak 6. In the same way, peak 6 in the L-GOS was also identified to be 6′-GL (See FIG. 1B).

The 6′-GL content in L-GOS was calculated by the peak percentage of 6′-GL of the total GOS (excluding the allolactose), as shown in Table 1.

TABLE 1
Composition of L-GOS and its 6′-GL content

| | | Peak percentage |
|---|---|---|
| Peak no | Peak Name | (%) |
| 1 | Galactose | 10.32 |
| 2 | Glucose | 19.4 |
| 3 | Allolactose | 14.07 |
| 4 | Lactose | 8.18 |
| 5 | Lactulose | 1.1 |
| 6 | 6′-galactosyl-lactose | 17.12 |
| | GOS (excluding allolactose) | 46.93 |
| | 6′-GL (% on GOS excl. allolactose) | 36.5 |

To determine the GOS Degree of polymerization (DP), oligosaccharides were separated using ion-exchange chromatography on a Rezex RSO column from Phenomenex, which has a high resolution for oligosaccharide till approximately DP18 (Degree of Polymerization). After the separation on the column the different components are measured with a RI detector. This detector is able to detect compounds on basis of refractive index. The individual DP percentage is calculated by the respective peak percentage. Table 2 shows the DP composition of an L-GOS composition according to the invention compared to reference composition Vivinal®GOS.

TABLE 2
DP composition of L-GOS and Vivinal ®GOS

| DP composition on Dry matter | | | DP composition on GOS (%) | | |
|---|---|---|---|---|---|
| DP | | Vivinal- | DP | | |
| composition | L-GOS | GOS | composition | L-GOS | Vivinal-GOS |
| DP7 | 0.32 | 0.73 | DP7 | 0.67 | 1.25 |
| DP6 | 0.26 | 1.37 | DP6 | 0.54 | 2.35 |
| DP5 | 1.06 | 4.19 | DP5 | 2.21 | 7.17 |
| DP4 | 5.57 | 9.99 | DP4 | 11.62 | 17.11 |
| DP3 | 22.24 | 21.49 | DP3 | 46.38 | 36.80 |
| DP2-GOS | 18.5 | 20.63 | DP2-GOS | 38.58 | 35.33 |
| sum of GOS* | 47.95 | 58.4 | | | |
| DP2 | 26.68 | 36.81 | DP2 | | |
| Lactose | 8.18 | 16.18 | lactose | | |
| Glucose | 22.07 | 18.89 | Glucose | | |
| Galactose | 21.8 | 0.33 | Galactose | | |

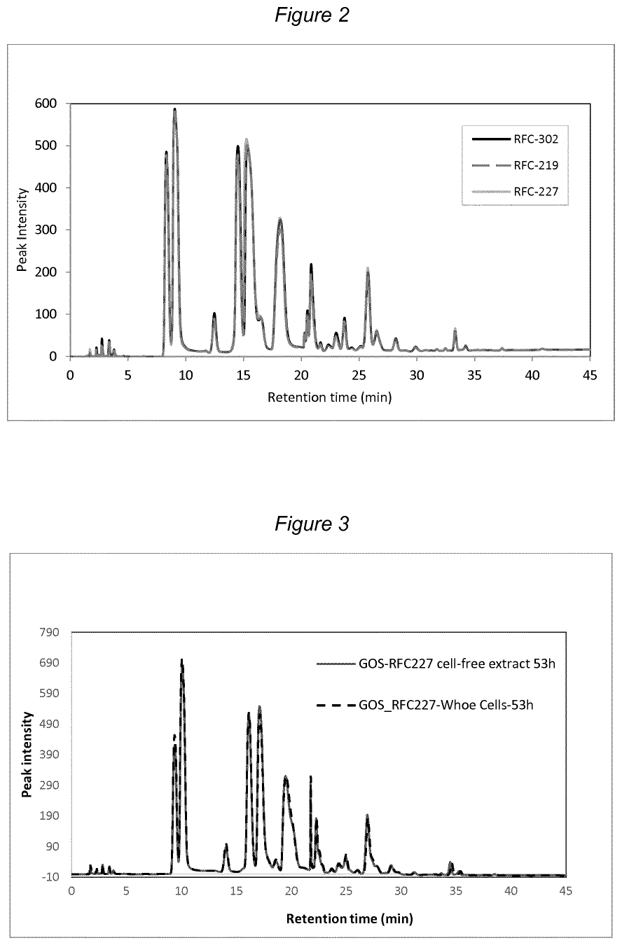

The GOS profiles obtained with the 3 whole cells are depicted in FIG. 2, which shows that the GOS profiles synthesized with each of the 3 enzyme preparations is actually identical, suggesting that the beta-galactosidases associated with these 3 Lactobacillus strains are functionally identical.

In view of this similarity, subsequent experiments were performed with whole cells and cell-free extract of strain RFC219 only, at an enzyme dosage of 3.3 LU/gram lactose used under the same conditions as mentioned above except with lactose concentration of 50% (w/w). Strain RFC219 is a Lactobacillus delbrueckii subsp. bulgaricus. The beta-galactosidase obtained from RFC219 has an amino acid sequence according to SEQ ID NO:1. An aliquot of 2.0 ml sample were taken at reaction time of 29 h, 36 h, 53 h and 65 hour and deactivated by addition of 1.5% 1.5 M HCl (v/v). The GOS composition and fingerprint profile were analyzed and summarized in Table 3 and FIG. 3. As expected, the GOS profiles by using whole cells or the isolated cell-free enzyme completely match, suggesting that the enzymatic kinetics are identical.

TABLE 3
GOS content and sugar analysis by Dionex HPAEC-PAD

| | | | Allo- | | | |
|---|---|---|---|---|---|---|
| Sample | Galactose | Glucose | lactose | Lactose | Lactulose | GOS* |
| GOS reference | 1.85 | 17.04 | 4.52 | 16.25 | 1.18 | 59.16 |
| Cell Free | 7.67 | 17.31 | 13.09 | 20.6 | 1.31 | 40.02 |
| extract -29 h | | | | | | |
| Cell Free | 7.84 | 17.14 | 13.03 | 19.18 | 1.17 | 41.64 |
| extract -36 h | | | | | | |
| Cell Free | 8.74 | 18.12 | 13.78 | 17.49 | 1.02 | 40.85 |
| extract -53 h | | | | | | |
| Cell Free | 10.32 | 19.4 | 14.07 | 8.18 | 1.1 | 46.93 |
| extract -65 h | | | | | | |
| Whole cell | 7.69 | 17.09 | 12.98 | 19.27 | 1.26 | 41.71 |
| synthesis-29 h | | | | | | |
| Whole cell | 8.37 | 17.52 | 13.34 | 18.3 | 0.88 | 41.59 |
| synthesis-36 h | | | | | | |
| Whole cell | 9.43 | 18.77 | 13.77 | 15.79 | 0.93 | 41.31 |
| synthesis-53 h | | | | | | |

GOS = 100 − galactose % − glucose % − allolactose % − lactose % − lactulose % (AOAC method)

### Example 4: Bifidogenic Effect of GOS Compositions

Partial purification of GOS by removing the mono-sugars was performed using a published method to Rodriguez-Colinas et al. (2013, Appl. Microbiol. And Biotechn. Vol. 97, pp 5743-5752).

A partially purified GOS preparation (“L-GOS test composition”) with the composition shown in Table 2, was tested for its bifidogenic effect using baby feces in an established in vitro model.

TABLE 4
Composition of partially purified L-GOS

| Component | Concentration (%) |
|---|---|
| galactose | 1.64 |
| Glucose | 4.07 |
| allo-lactose | 14.82 |
| Lactose | 6.86 |
| lactulose | 0.48 |
| GOS | 72.13 |
| GOS + allolactose | 86.95 |

The bifidogenic effect of GOS was evaluated using TIM-2 model (TNO in vitro model of the colon (TIM-2), Venema K. (2015), The TNO In Vitro Model of the Colon (TIM-2). In: Verhoeckx K. et al. (eds), The Impact of Food Bioactives on Health. Springer, Cham), which is able to simulate material passing the ileo-caecal valve in humans. The microbiota was fed into the system through a food syringe, which contains a simulated ileal efflux medium (SIEM).

The microbiota used in this model for the current invention was established via fecal donations from 6 healthy infants (between 1-6 months old, bottle fed and no use of antibiotics for at least one month prior donation). Moreover, all babies were predominately bottle-fed. Since the feces of baby 4 did not contain any detectable bifidogenic activity, this sample was withdrawn from the assay.

The standard medium used contained the following components (g): pectin (9.4), xylan (9.4), arabinogalactan (9.4), amylopectin (9.4), casein (47.0), starch (78.4), Tween 80 (34.0), Bacto Peptone (47.0) and ox bile (0.8). Dialysis liquid contained (per litre): 2.5 g K₂HPO₄.3H₂O, 4.5 g NaCl, 0.005 g FeSO₄.7H₂O, 0.5 g MgSO₄.7H₂O, 0.45 g CaCl₂.2H₂O, 0.05 g bile and 0.4 g cysteine.HCl, plus 1 ml of a vitamin mixture containing (per litre): 1 mg menadione, 2 mg D-biotin, 0.5 mg vitamin B12, 10 mg pantothenate, 5 mg nicotinamide, 5 mg p-aminobenzoic acid and 4 mg thiamine.

To determine the bifidogenic effect, the total carbohydrate was equivalently substituted by either a sugar control, the L-GOS test composition of the invention or Reference compositions GOS1 and GOS2. The sugar control is a composition equivalent to the sugar composition of the mono sugars (galactose and glucose plus lactose present in the corresponding purified GOS preparation). GOS1 and GOS2 refer, respectively, to GOS prepared with a beta-galactosidase from Bifidobacteria longum and the commercial product Vivinal®GOS.

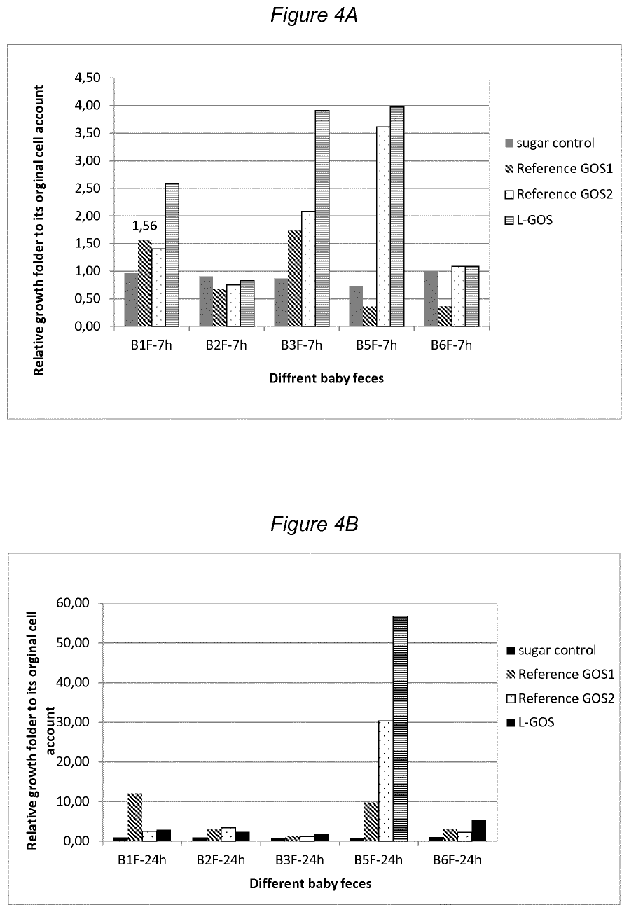

The Bifidobacterium growth rate was analyzed after 7 hours (FIG. 4A) and 24 hours (FIG. 4B).

As shown in FIG. 4, the L-GOS composition of the invention is able to stimulate the growth of the 5 baby's feces most effectively when compared to either the sugar control or the reference compositions GOS1 and 2.

### Example 5: Hypoallergenicity of an Oligosaccharide Compositions of the Invention

This example demonstrates the reduced allergenicity of an oligosaccharide composition of the invention in four human subjects with known galacto-oligosaccharide allergy. L-GOS obtained by transgalactosylation using beta-galactosidase from strain RFC227 cell-free extract and a commercial GOS reference preparation obtained using B. circulans enzyme (vGOS) were included in the test.

Eligible subjects were selected from the cohort previously studied for the prevalence of GOS-allergy in a Singapore atopic population, as described by Soh et al., (Allergy 2015, 70, 1020-3).

A Basophil Activation Test (BAT) was performed on patient blood samples. To that end, heparinized peripheral blood aliquots (100 μL) were pre-incubated at 37° C. for 5 minutes and then incubated with 100 μL of PBS (negative control), anti-IgE antibody (positive control, G7-18; BD Biosciences, San Jose, Calif) or diluted GOS samples for 15 minutes (37° C.). After incubation, cells were washed in PBS-EDTA (20 mmol/L) and then incubated with phycoerythrin-labeled anti-human IgE (Ige21; eBioscience, San Jose, Calif), biotin-labeled anti-human CD203c (NP4D6; BioLegend, San Jose, Calif), and fluorescein isothiocyanate-labeled anti-human CD63 (MEM-259, BioLegend) mAbs for 20 minutes at 48° C. Expression of CD203c and CD63 are both markers for basophil activation.

After washing the cells with 1% BSA/PBS, allophycocyanin-conjugated streptavidin (BD Biosciences) was added and incubated for 15 minutes at 48° C. Thereafter, samples were subjected to erythrocyte lysis with 2 mL of FACS Lysing Solution (BD Biosciences). Cells were then washed, resuspended in 1% BSA/PBS, and analysed by means of FACSCalibur (BD Biosciences). Basophils were detected on the basis of side-scatter characteristics and expression of IgE (IgEhigh).

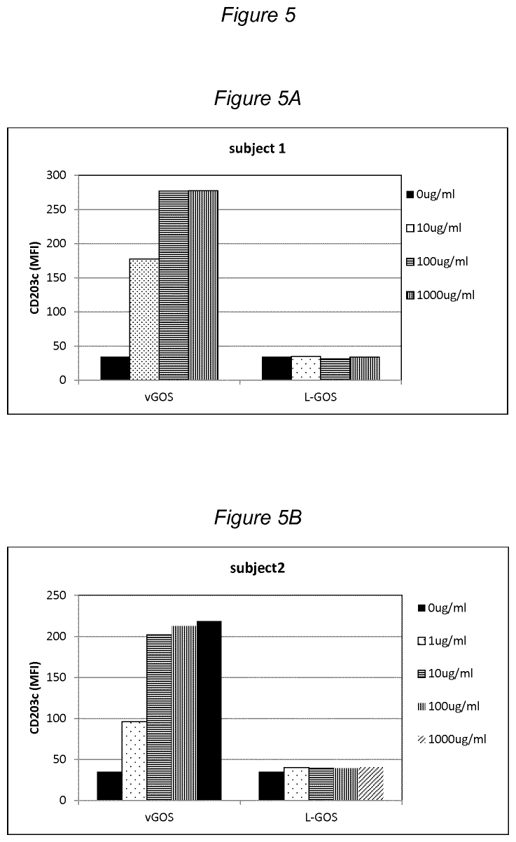

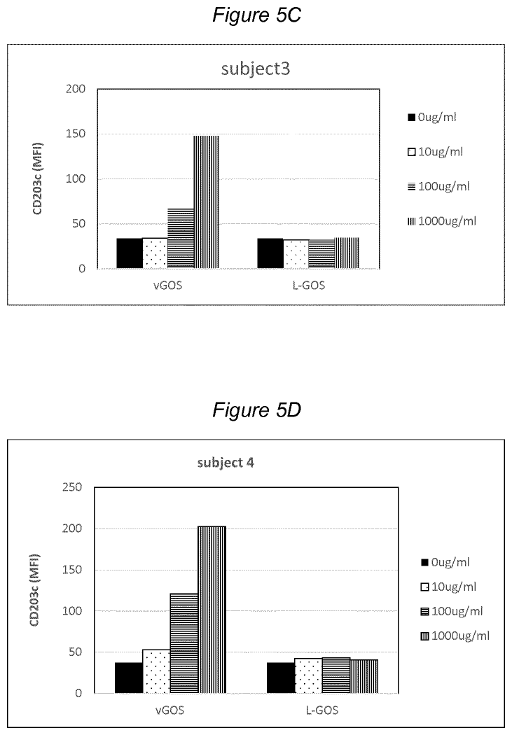

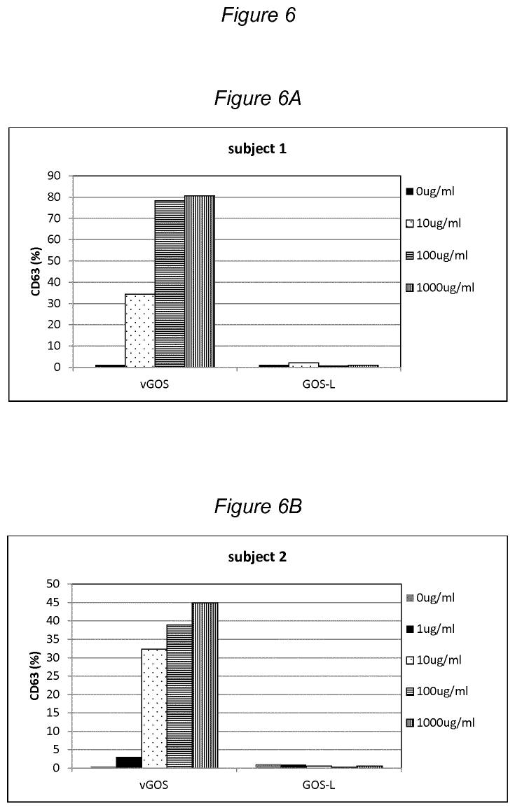

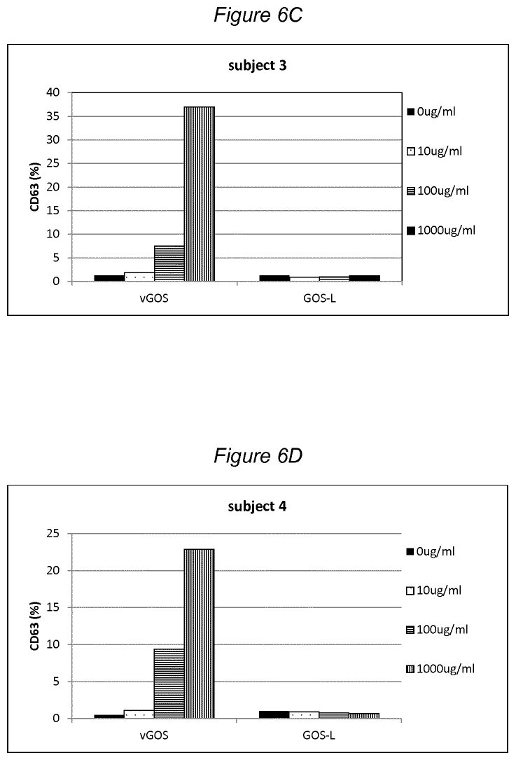

In contrast to the reference GOS composition, L-GOS prepared with Lactobacillus enzyme of the strains used in the present invention elicits no positive reaction in BAT test, as evidenced by the very low or virtually no expression of the activation markers CD203c (FIG. 5) and CD63 (FIG. 6).

### Example 6: Determination of the Gene and Protein Sequence of the Lactobacillus Enzymes

An attempt was made to determine the gene sequence encoding the ß-galactosidase produced by the three Lactobacillus delbreuckii strains.

Bacteria were cultured in MRS liquid media as mentioned above to reach a OD of ˜1.0. The DNA extraction was performed directly with this biomass. The DNA extraction protocol used is mainly based on the use of Zymo Research Bacterial/Fungal DNA microPrep kit D6007. The obtained DNA sample was analysed by the Illumina HiSeq2500.

Quality Analysis of FASTQ Sequence Reads

Paired-end sequence reads were generated using the Illumina HiSeq2500 system. FASTQ sequence files were generated using bcl2fastq2 version 2.18. Initial quality assessment was based on data passing the Illumina Chastity filtering. Subsequently, reads containing PhiX control signal were removed using an in-house filtering protocol. In addition, reads containing (partial) adapters were clipped (up to minimum read length of 50 bp. The second quality assessment was based on the remaining reads using the FASTQC quality control tool version 0.11.5.

De Novo Assembly

Assembly

The quality of the FASTQ sequences was enhanced using the read error correction module BayesHammer in the SPAdes version 3.10 genome assembly toolkit (Bankevich A et. Al. (2012)J Comput Biol. 19:455-477) The high-quality reads were assembled into contigs using SPAdes. Misassemblies and nucleotide disagreement between the Illumina data and the contig sequences are corrected with Pilon (Walker B J et. al. (2014) PLOS ONE 9(11): e112963) version 1.21.

Scaffolding

The contigs were linked and placed into scaffolds, where the orientation, order and distance between them were estimated using the insert size between the paired-end and/or matepair reads. The analysis has been performed using the SSPACE Premium Scaffolder version 2.3 (Boetzer et. al, 2011).

Automated Gap Closure

The gapped regions within the scaffolds are (partially) closed in an automated manner using GapFiller version 1.10 (Boetzer and Pirovano, 2012). The method takes advantage of the insert size between the paired-end and/or matepair reads.

The obtained genome sequences were annotated using DNA annotation tool: “ClustalW”(https://www.genome.jp/tools-bin/clustalw), the conversion of DNA to protein was done in an offline package (“sms2”, downloaded from http://www.bioinformatics.org/sms2/).

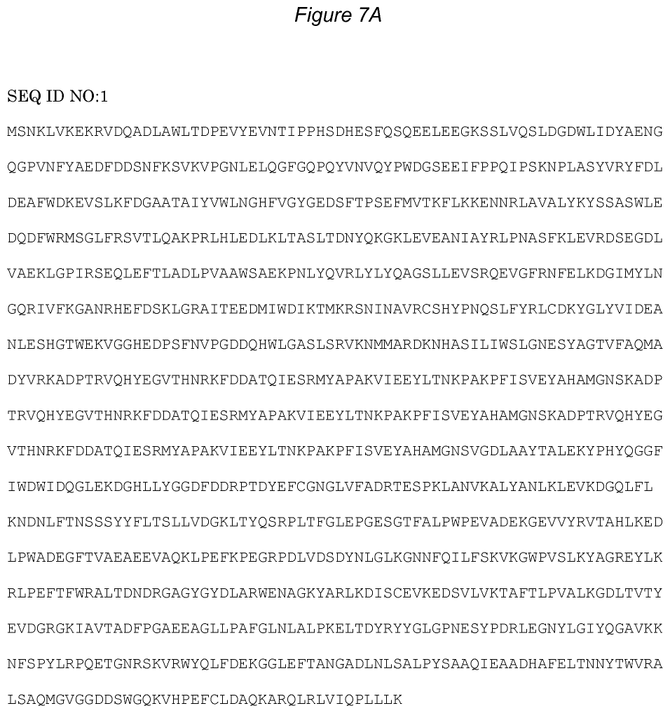

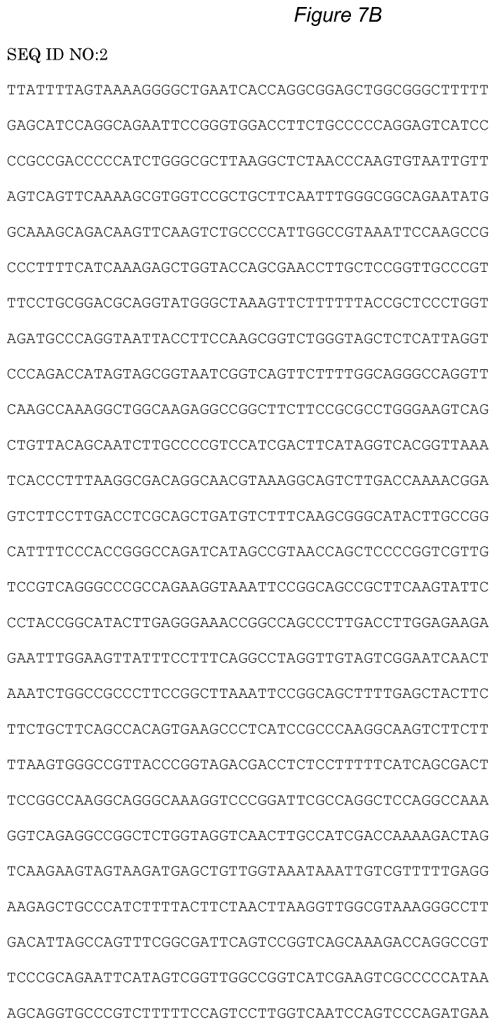

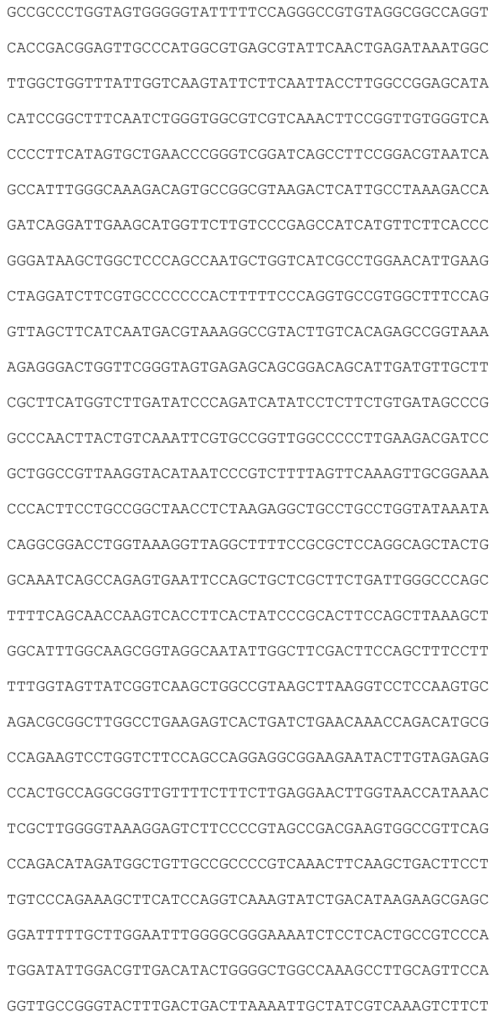

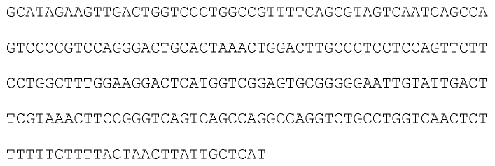

The amino acid sequence and the nucleotide sequences of a representative ß-galactosidase obtained are shown in FIGS. 7A and 7B, respectively.

1. An oligosaccharide composition comprising:
(i) at least 40% galacto-oligosaccharides (GOS) by weight of the total dry matter of the composition;
(ii) at least 10% allolactose by weight of the total dry matter of the composition;
(iii) at least 30% 6′-galactosyl-lactose (6′-GL) by weight of the total GOS in the composition; and
(iv) at least 0.5% by weight of the total GOS has a polymerization degree (DP) of six or more.

2. The oligosaccharide composition of claim 1, wherein the GOS content is at least 42% by weight of the total dry matter of the composition.

3. The oligosaccharide composition of claim 1, wherein at least 1% by weight of the total GOS has a DP of six or more.

4. The oligosaccharide composition of claim 1, wherein the allolactose content is at least 12% by weight of the total dry matter of the composition.

5. The oligosaccharide composition of claim 1, wherein the 6′-GL content is at least 40% by weight of the total GOS in the composition.

6. The oligosaccharide composition of claim 1, wherein
(i) the GOS content is at least 70% by weight of the total dry matter of the composition;
(ii) the allolactose content is at least 12% by weight of the total dry matter of the composition;
(iii) the 6′-GL content is at least 40% by weight of the total GOS in the composition; and
(iv) at least 1% by weight of the total GOS has a polymerization degree (DP) of six or more.

7. A nutritional composition comprising the oligosaccharide composition of claim 1, wherein said nutritional composition is an infant formula, a follow-up formula, a growing-up milk, a dairy product, a cereal, or a medical nutritional product.

8. A nutritional composition comprising protein source, a lipid source, a carbohydrate source, and the oligosaccharide composition of claim 1.

9. (canceled)

10. A method of promoting gut microbiota balance and health in a subject, the method comprising administering to the subject an effective amount of the oligosaccharide composition of claim 1.

11. The method of claim 10, wherein said promoting gut microbiota and health comprises enhancing bifidogenic micro-organisms in the intestinal tract and/or improving patient tolerance to at least one medical treatment that leads to a gastrointestinal tract disorder.

12. The oligosaccharide composition of claim 1, wherein the oligosaccharide composition has prebiotic properties effective to promote gut microbiota balance and health in an intestinal tract of a human or animal.

13. A method of making a bifidogenic infant formula or dietetic food, the method comprising adding the oligosaccharide composition of claim 1 to a component selected from the group consisting of fats, carbohydrates, minerals, trace elements, vitamins, and combinations thereof.

14. A method of making the oligosaccharide composition of claim 1, the method comprising (i) contacting a lactose feed with a beta-galactosidase (EC 3.2.1.23), and (ii) allowing for oligosaccharide synthesis, wherein said beta-galactosidase has an amino acid sequence having at least 98% sequence identity to SEQ ID NO:1.

15. The method of claim 14, wherein said beta-galactosidase is contacted with said lactose feed while being present in a micro-organism endogenously expressing said beta-galactosidase, wherein said micro-organism is used as whole cells or an active part or fraction thereof, and the micro-organism is Lactobacillus delbrueckii subspecies bulgaricus strain DSM20080.

16. The oligosaccharide composition of claim 1, wherein the GOS content is at least 60% by weight of the total dry matter of the composition.

17. The oligosaccharide composition of claim 1, wherein at least 1.5% by weight of the total GOS has a DP of six or more.

18. The oligosaccharide composition of claim 1, wherein the 6′-GL content is at least 43% by weight of the total GOS in the composition.

19. The method of claim 11, wherein the at least one medical treatment is selected from the group consisting of radiotherapy, chemotherapy, gastrointestinal surgery, anesthesia, the administration of antibiotics, analgesic drugs, and treatment for diarrhea.

20. The method of claim 14, wherein the beta-galactosidase (EC 3.2.1.23) is obtained from Lactobacillus delbrueckii subspecies bulgaricus strain DSM20080.


SEQUENCE LISTING (2 sequences)

SEQ ID NO: 1 - protein, 1128 aa, Lactobacillus delbrueckii
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSKADPTRVQHYEGVTHNRKFDDATQIESRMYAPAKVIEEYLTNKPAKPFISVEYAHAMGNSKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 2 - DNA, 3027 bp, Lactobacillus delbrueckii
ttattttagtaaaaggggctgaatcaccaggcggagctggcgggctttttgagcatccaggcagaattccgggtggaccttctgcccccaggagtcatccccgccgacccccatctgggcgcttaaggctctaacccaagtgtaattgttagtcagttcaaaagcgtggtccgctgcttcaatttgggcggcagaatatggcaaagcagacaagttcaagtctgccccattggccgtaaattccaagccgcccttttcatcaaagagctggtaccagcgaaccttgctccggttgcccgtttcctgcggacgcaggtatgggctaaagttcttttttaccgctccctggtagatgcccaggtaattaccttccaagcggtctgggtagctctcattaggtcccagaccatagtagcggtaatcggtcagttcttttggcagggccaggttcaagccaaaggctggcaagaggccggcttcttccgcgcctgggaagtcagctgttacagcaatcttgccccgtccatcgacttcataggtcacggttaaatcaccctttaaggcgacaggcaacgtaaaggcagtcttgaccaaaacggagtcttccttgacctcgcagctgatgtctttcaagcgggcatacttgccggcattttcccaccgggccagatcatagccgtaaccagctccccggtcgttgtccgtcagggcccgccagaaggtaaattccggcagccgcttcaagtattccctaccggcatacttgagggaaaccggccagcccttgaccttggagaagagaatttggaagttatttcctttcaggcctaggttgtagtcggaatcaactaaatctggccgcccttccggcttaaattccggcagcttttgagctacttcttctgcttcagccacagtgaagccctcatccgcccaaggcaagtcttcttttaagtgggccgttacccggtagacgacctctcctttttcatcagcgacttccggccaaggcagggcaaaggtcccggattcgccaggctccaggccaaaggtcagaggccggctctggtaggtcaacttgccatcgaccaaaagactagtcaagaagtagtaagatgagctgttggtaaataaattgtcgtttttgaggaagagctgcccatcttttacttctaacttaaggttggcgtaaagggccttgacattagccagtttcggcgattcagtccggtcagcaaagaccaggccgttcccgcagaattcatagtcggttggccggtcatcgaagtcgcccccataaagcaggtgcccgtctttttccagtccttggtcaatccagtcccagatgaagccgccctggtagtgggggtatttttccagggccgtgtaggcggccaggtcaccgacggagttgcccatggcgtgagcgtattcaactgagataaatggcttggctggtttattggtcaagtattcttcaattaccttggccggagcatacatccggctttcaatctgggtggcgtcgtcaaacttccggttgtgggtcaccccttcatagtgctgaacccgggtcggatcagccttccggacgtaatcagccatttgggcaaagacagtgccggcgtaagactcattgcctaaagaccagatcaggattgaagcatggttcttgtcccgagccatcatgttcttcacccgggataagctggctcccagccaatgctggtcatcgcctggaacattgaagctaggatcttcgtgcccccccactttttcccaggtgccgtggctttccaggttagcttcatcaatgacgtaaaggccgtacttgtcacagagccggtaaaagagggactggttcgggtagtgagagcagcggacagcattgatgttgcttcgcttcatggtcttgatatcccagatcatatcctcttctgtgatagcccggcccaacttactgtcaaattcgtgccggttggcccccttgaagacgatccgctggccgttaaggtacataatcccgtcttttagttcaaagttgcggaaacccacttcctgccggctaacctctaagaggctgcctgcctggtataaatacaggcggacctggtaaaggttaggcttttccgcgctccaggcagctactggcaaatcagccagagtgaattccagctgctcgcttctgattgggcccagcttttcagcaaccaagtcaccttcactatcccgcacttccagcttaaagctggcatttggcaagcggtaggcaatattggcttcgacttccagctttcctttttggtagttatcggtcaagctggccgtaagcttaaggtcctccaagtgcagacgcggcttggcctgaagagtcactgatctgaacaaaccagacatgcgccagaagtcctggtcttccagccaggaggcggaagaatacttgtagagagccactgccaggcggttgttttctttcttgaggaacttggtaaccataaactcgcttggggtaaaggagtcttccccgtagccgacgaagtggccgttcagccagacatagatggctgttgccgccccgtcaaacttcaagctgacttccttgtcccagaaagcttcatccaggtcaaagtatctgacataagaagcgagcggatttttgcttggaatttggggcgggaaaatctcctcactgccgtcccatggatattggacgttgacatactggggctggccaaagccttgcagttccaggttgccgggtactttgactgacttaaaattgctatcgtcaaagtcttctgcatagaagttgactggtccctggccgttttcagcgtagtcaatcagccagtccccgtccagggactgcactaaactggacttgccctcctccagttcttcctggctttggaaggactcatggtcggagtgcgggggaattgtattgacttcgtaaacttccgggtcagtcagccaggccaggtctgcctggtcaactcttttttcttttactaacttattgctcat